(19) United States
(12) Patent Application Publication
(10) Pub. No.: US 2010/0130850 A1
(43) Pub. Date: May 27, 2010

(54) Flexible Core Surgical Device

(76) Inventor: Robert L. Pakter, Tucson, AZ (US)

Correspondence Address: FISH & RICHARDSON P.C., PO BOX 1022, MINNEAPOLIS, MN, 55440-1022, US

(21) Appl. No.: 12/626,386

(22) Filed: Nov. 25, 2009

Related U.S. Application Data
(60) Provisional application No. 61/117,953, filed on Nov. 25, 2008.

(51) Int. Cl. A61B 5/055; A61B 10/02

(52) U.S. Cl. 600/411; 600/567

(57) ABSTRACT

A surgical instrument having a tubular member having a proximal end and a distal end, at least one helical member concentric with the tubular member, and a cutting portion protruding from the distal end of the tubular member. A method of using the surgical instrument is performing a biopsy.

### CLAIM OF PRIORITY

This applications claims the benefit of U.S. Provisional Application No. 61/117,953, filed Nov. 25, 2008, which is incorporated herein by reference.

### FIELD

The present disclosure relates generally to a surgical instrument for performing biopsies, more specifically to a surgical instrument that is both nonferromagnetic and kink resistant.

### BACKGROUND

Magnetic resonance imaging (MRI) is a well-known and useful technique for imaging the human body. It is particularly useful as a non-invasive procedure that can be used without exposing biological tissue to the potentially damaging effects of radiation. Recent advances in MRI technology, such as receiver coil design, have enabled imaging methodologies previously unforeseen. One particular advance is the ability to perform endoluminal imaging, which provides images inside blood vessels and other tubular structures such as the urethra, bronchi, and bowel. Such imaging can be useful in evaluating, diagnosing, and treating a number of clinical conditions, such as various types of cancer and atherosclerosis.

Although advances have been made in MRI imaging, treatments are sometimes difficult, as tools have not yet been developed that are appropriate for use with the latest imaging techniques. Traditional surgical instruments and materials can disrupt an MRI due to their ferromagnetic properties. Furthermore, these tools may be too large or rigid, preventing them from reaching regions of the body that are not directly accessible, e.g., regions that are not located along a straight-line axis from a point of insertion.

### SUMMARY

In general, in one aspect, the technology herein includes a surgical instrument including a tubular member having a proximal end and a distal end, at least one helical member concentric with the tubular member, and a cutting portion protruding from the distal end of the tubular member.

In another aspect, the technology herein includes a method of performing a biopsy including inserting a surgical instrument into a region of the body, rotating the surgical instrument along an axis perpendicular to a radius of the tubular member such that the cutting portion cuts a sample of a tissue, and withdrawing the surgical instrument such that the sample is removed from the region of the body. The surgical instrument includes a tubular member having a proximal end and a distal end, at least one helical member concentric with the tubular member, and a cutting portion protruding from the distal end of the tubular member.

Certain implementations may have one or more of the following advantages. The nonferromagnetic properties of the surgical instrument can allow the surgical instrument to be used in conjunction with monitoring a procedure by MRI. Moreover, the materials of the core can be chosen such that the core can be bent from an initial straight line to an alternative configuration to reach otherwise inaccessible areas of the body.

The details of one or more embodiments of the invention are set forth in the accompanying drawings and the description herein. Other features, aspects, and advantages of the invention will become apparent from the description, the drawings, and the claims.

### BRIEF DESCRIPTION OF THE DRAWINGS

FIG. 1 shows an embodiment of a core of a surgical device as described herein.

FIG. 2 shows a cross-section of an embodiment of a surgical instrument described herein.

FIG. 3 shows an embodiment of an imaging device described herein.

FIG. 4 shows cross-section of an embodiment of an imaging device and tubular guide channel as described herein.

FIG. 5 shows a schematic of the placement of a surgical instrument within the urethra and prostate.

FIG. 6A shows an illustration of the placement of a sheath and dilator for insertion of a surgical instrument.

FIG. 6B shows an illustration of the removal of a dilator and guidewire for insertion of a surgical instrument.

FIG. 6C shows an illustration of the placement of an imaging device described herein within a sheath.

FIG. 7 shows a cross-section of an illustration of the placement of a tubular guide channel with a sharp tip as described herein.

FIG. 8 is a schematic representation of the insertion of a core as described herein.

Like reference numbers and designations in the various drawings indicate like elements.

### DETAILED DESCRIPTION

The surgical device described herein typically comprises a tubular member with a proximal end and a distal end and at least one helical member concentric with the tubular member. An end portion can protrude from the distal end of the tubular member and may be fashioned with a desired configuration, such as a saw tooth pattern to enable cutting.

As used herein, the terms proximal and distal correspond with the manner in which they are customarily used in medicine and surgery, i.e., the proximal portion of an apparatus is that which resides—during operation—closer to the user, operator, or surgeon, than the remainder of the apparatus. Conversely, the distal portion of an apparatus is that which—during operation is situated closer to the patient or subject than the remainder of the apparatus.

### Device, Generally

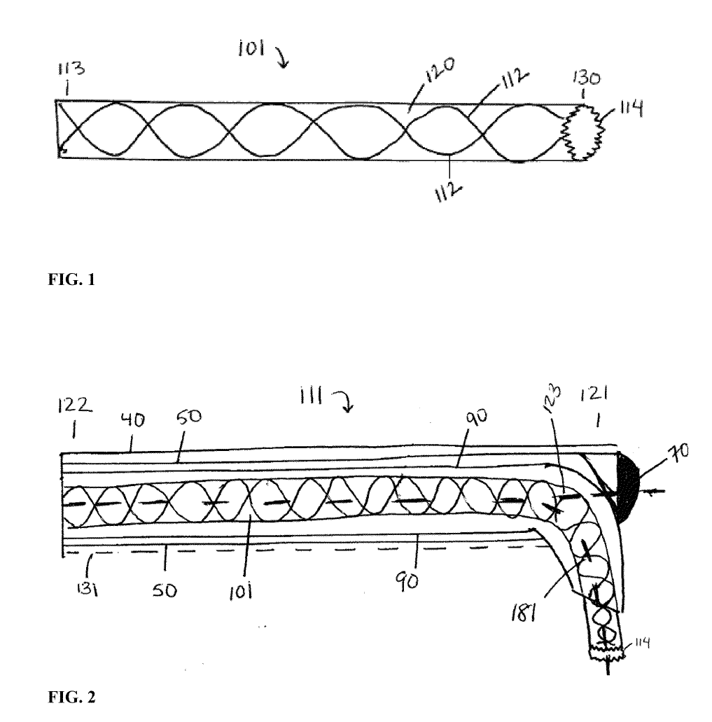

Referring to FIG. 1, the core 101 includes a hollow tube 120 and helical members 112. The helical members 112 can be embedded in the periphery of the hollow tube 120. The hollow tube 120 can be composed of a polymer, such as moderately rigid polyurethane or polytetrafluoroethylene, and can be 3 to 6 French (1 mm to 2 mm) in dimension. The helical members 112 can be made of a metal alloy, such as a nickel titanium alloy, e.g., Nitinol. Use of Nitinol is advantageous in that it is both non-ferromagnetic and kink resistant. Other alloys having similar properties are consistent with the technology described herein.

Each helical member 112 can run the length of the hollow tube 120, but may run for only a portion of the length. Although FIG. 1 shows only two helical members 112, there can be a greater number of helical members, such as, three, four, five, or six, or more. In another embodiment, not shown, a single helical member is deployed. The pitch of each helix may be narrow, e.g. a sinusoidal pattern, or wide. In another embodiment, the helical members 112 can be arranged as two reciprocating double helices, e.g., as anterior and posterior helices that are not fused in the center line.

The helical members 112 can be arranged in an asymmetrical weave such that the density of metal is greatest at the distal end 130 of the core, i.e. the pitch of the helix can become progressively narrower approaching the distal end 130 of the core. Such an arrangement can result in greater malleability at the proximal end 113 of the core where bending may be desired and greater stiffness at the distal end 130 where a more rigid structure may be required to, for example, cut tissue.

In one embodiment, the helical members 112 can be formed by obtaining a single solid tube of a metal alloy, such as Nitinol. The solid tube can then be laser cut into a desired pattern, i.e., portions can be etched away until reciprocating helical members 112 remain. In another embodiment, the helical members can be formed through the use of a mold. The hollow tube 120 can then be formed around the helical members 112. For example, helical members 112 can be placed into an extrusion mold to serve as scaffolding. A plastic such as polyurethane or polytetrafluoroethylene can then be poured over the helical members 112.

In one embodiment, the ends of the helical members 112 located at the distal end 130 of the hollow tube 120 can be contiguous with an annular concentric member 114 protruding from the distal tip of the catheter. The annular concentric member 114 can be only at the distal end 130 of the core 101 and can be made of the same material as the helical members 112, e.g., of a nickel titanium alloy such as Nitinol. The helical members 112 can meld together to form the annular concentric member 114. Alternatively, the annular concentric member 114 can be made of a different material than the helical members 112. The annular concentric member 114 can be fashioned to have sharp edges at the distal end 130, e.g., can be configured in a saw-tooth manner, as shown in FIG. 1. Alternatively, the annular concentric member 114 may be undulating and obtuse. The solid member may also be machined, engineered, or fashioned to have a distal end configured to perform a number of surgical operations, including ablating, coring, piercing, macerating, dissecting, and cutting.

In another embodiment, the ends of the helical members 112 located at the distal end 130 of the hollow tube 120 can extend beyond the hollow tube 120 and can be configured to be sharp or undulating and obtuse.

There can be a non-ferromagnetic luer connection (not shown) at the proximal end 113 of the core 101. The luer connection can permit attachment to a hand-held rotational drive device (not shown). A radio frequency (RF) generator may be a component of the drive device. Alternatively, an RF generator may supplant the drive device.

During operation, as will be discussed further herein, when the drive device is activated, the core 101 can spin about an axis longitudinal to the hollow tube 120. The sharp edges of the annular concentric member 114 can rotate and act as a rotary saw to cut samples of tissue in the body. In other embodiments, the annular concentric member 114 may macerate clot as a thrombectomy device. In yet another embodiment, an obtuse end may serve a an ablative device using RF energy.

### Device With Layers

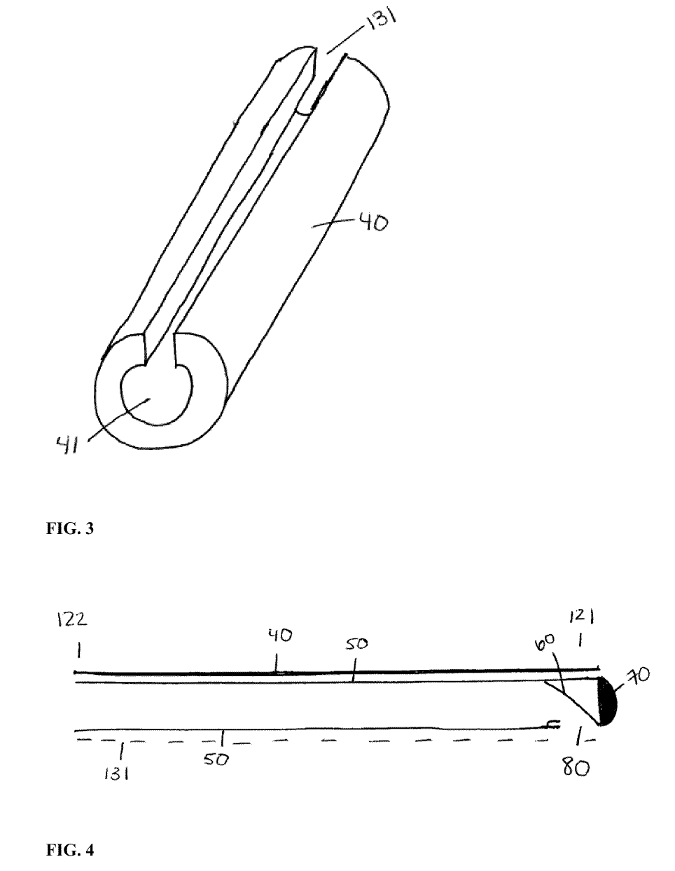

Referring to FIG. 2, the surgical instrument 111 can include a magnetic resonance imaging device 40, such as a quadrature coil or an ultrasound device. The imaging device 40 can include imaging hardware (not shown) along its perimeter. The imaging device can serve as the receiver coil for signals generated by tissues and can be subjected to radiofrequency gradients in an MRI magnet. The imaging device 40 can also serve as a platform for an interventional procedure such that a second device may be coaxially advanced to a set tissue point and a surgical procedure performed. The imaging device 40 can have an outer tube or longitudinal working channel 41 (shown in FIG. 3) along its inner diameter. The working channel 41 can be part of the imaging device 40 or can be a distinct tube made of, for example, a high density plastic such as polycarbonate or a highly buffed metal such as an alloy of nickel and titanium. The working width of the working channel 41 can be wide enough to accommodate a 16 gauge to 11 gauge needle (1.651 mm to 1.048 mm), such that it may accept a catheter 4 to 10 French in size (1.35 mm to 3.3 mm). The imaging device 40 can include an aperture or slit 131 (see FIG. 3 and dotted line in FIG. 2) running from the proximal end 122 to the distal end 121 of the device. As discussed further herein, the slit 131 allows access to the tissue from any point along the working channel 41. In other embodiments, a slit runs from the distal end to an intermediate location along the length of the device, and in still other embodiments a slit runs between two points interior to the proximal and distal ends of the device.

Referring still to FIG. 2, an inner tube or tubular guide channel 50 can be located within the working channel 41 (see FIG. 3) of the imaging device 40. The tubular guide channel 50 can be a hollow tube made of, for example, a high density plastic such as polycarbonate or a highly buffed metal such as an alloy of nickel and titanium. There can be a short egress aperture 80 (see FIG. 4) in the tubular guide channel 50 near the distal end 121 of the surgical instrument 111. The aperture 80 can be of sufficient length to accommodate the passage of a malleable needle 90, as discussed below. Aperture 80 in the tubular guide channel 50 can be aligned with slit 131 in the imaging device 40 (see FIG. 4). Discussed further herein, this alignment can allow aperture 80 to be advanced or withdrawn from the imaging device 40.

Referring to FIGS. 2 and 4, tubular guide channel 50 can include an end portion 70 at the distal end 121 of the surgical instrument. The end portion 70 can be composed of a soft material, such as an elastomeric material such as silicone. While not limited to such a material, use of the soft material can ensure that if the guide chamber 50 were advanced beyond the distal end of the imaging device 40, it would be unlikely to damage body tissue, such as the walls of the urethra or other nearby tissues. Other configurations of end portion 70 (not shown) that similarly would result in minimal damage to body tissue can also be envisaged, including, for example, a sprung flap at end portion 70, or a retractable or telescoping segment proximal to distal end portion 70. The distal end of the tubular guide chamber 50 can have a luminal negatively banked wall 60 that can be slanted at a negative angle, such as 45°. This angle can allow a biopsy device, such as a malleable needle, to deflect out of the aperture 80, as discussed further herein.

As shown in FIG. 2, the surgical instrument 111 can further include a malleable needle 90, such as a curved needle, a catheter, or a trocar. The malleable needle 90 can be inserted inside the tubular guide channel 50. The malleable needle can be composed of a superelastic material such as Nitinol and can measure approximately 11 gauge to 20 gauge. For example, a 19 or 20 gauge needle could fit coaxially within a 3 French hollow tube 120. A 14 or 15 gauge needle could fit coaxially within a 6 French hollow tube 120.

In one embodiment, the core 101 discussed elsewhere herein can be disposed within the malleable needle 90, as shown in FIG. 2. The core 101 can have a hydrophilic coating, such as polyvinylpyrrolidone, to reduce friction as it rotates within the malleable needle 90. In another embodiment, an alternate core such as a curved solid superelastic needle (not shown) can be disposed within the malleable needle 90.

The surgical instrument 111 can thus include a core 101 surrounded coaxially by a malleable needle 90. The malleable needle 90 can be coaxially surrounded by a tubular guide channel 50. The guide channel, in turn, can be coaxially surrounded by and attached to an imaging device 40.

### Biopsy Procedure

In one embodiment, the surgical instrument 111 can be used, for example, to biopsy a tissue located at a point off of the center-line axis 123 (see FIG. 2). FIG. 5 shows a schematic of the device 111 placed inside a urethra 189 and a region 151 of the prostate. Thus, the device 111 can be used to biopsy the region 151 of the prostate using a transurethral approach.

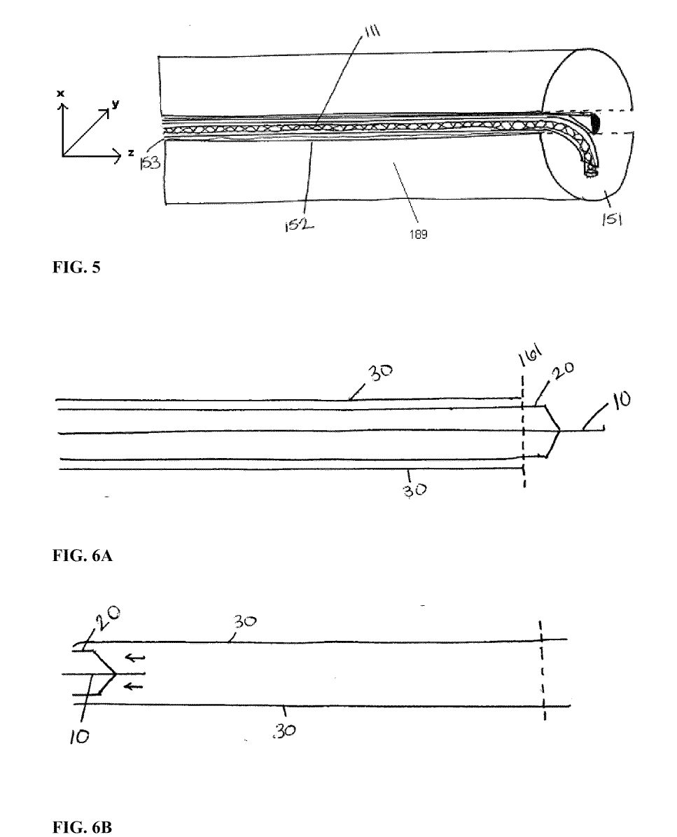

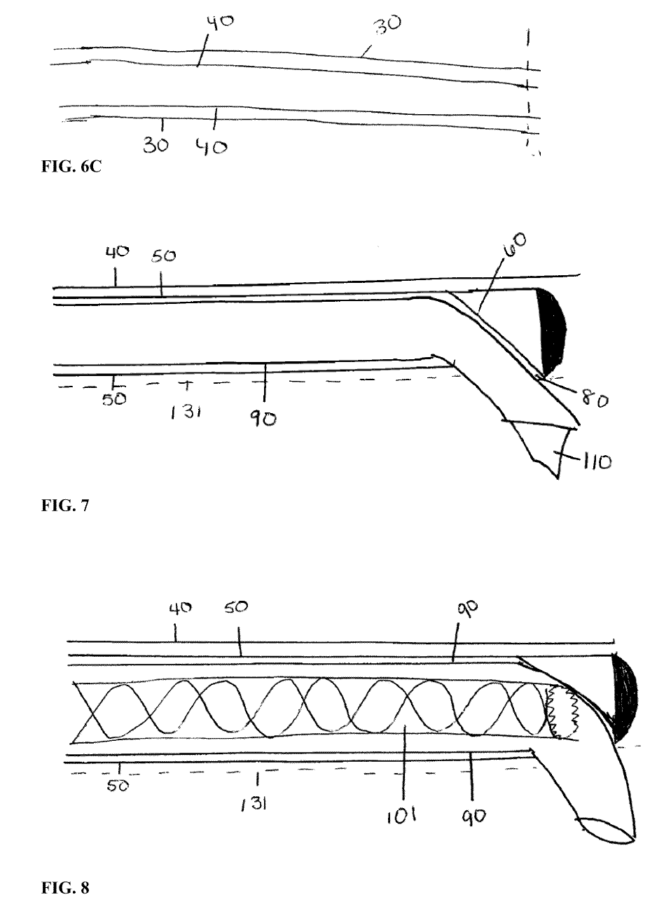

FIGS. 6A through 6C show how the sheath 30 is placed for later insertion of the surgical device 111. FIG. 7 shows the placement of the tubular guide channel using the preplaced sheath 30. Moreover, FIG. 8 shows the placement of the core 101 using the preplaced tubular guide channel 50.

During the biopsy procedure, the patient can be sedated. Regional anesthesia can be administered via, e.g., an injection of a local anesthetic, such as viscous lidocaine. The anesthesia can be injected, for example, through the urethral orifice 153. Referring to FIG. 6A, a lubricous medical guidewire 10 can be advanced into the body region of interest, for example, through the urethra 152 into the bladder (not shown).

As shown in FIG. 6A, a sheath 30, for example a sheath between 4 French (1.35 mm) and 10 French (3.3 mm), and dilator 20 system can be advanced over the wire until the dilator 20 is in the region of interest 161. Confirmation that the dilator 20 is in the correct position can be obtained through repeated imaging. As shown in FIG. 6B, the dilator 20 and guidewire 10 can then be removed. Referring to FIG. 6C, the imaging device 40 can then be advanced through the sheath 30 into the region of interest, for example the prostatic urethra. The length of the imaging device 40 can be sufficient to accommodate patients of various sizes, for example of varied urethral lengths. An external stabilization device (not shown) can ensure that there is no movement of the imaging device within the patient. The sheath 30 can then be removed.

Imaging of the tissue of interest can be performed. For example, imaging of the prostate can be performed from the imaging device 40 in the urethra. In another embodiment, lung tissue and lymph nodes adjacent to the central airways of a lung may be examined using imaging device 40. The tissue of interest may be lateral or oblique to the working lumen of the imaging device 40. The location of the tissue of interest, for example a prostate lesion, can be mapped by computer software (not shown). Coordinates can be defined by the circumferential locus of the tissue of interest relative to the working channel 41 in the xy-plane and the distance from the entry point, such as the urethral orifice 153 (see FIG. 5) along the z-axis.

Based on the data, a computer algorithm such as used elsewhere in the imaging arts, for example, can predict the coordinates for the placement of working channel 41 and tubular guide channel 50 and its aperture 80. The device coordinates can be based on the tissue of interest location and the parameters of the surgical instrument 111, including the curvature of the guide trocar needle 90. For example, using data obtained by imaging device 40, an abnormal locus in xyz coordinates can be identified. The imaging device 40 can then be rotated in the xy-plane such that slit 131 is aligned with the locus of the lesion. Guide device 50 can be advanced to the appropriate locus in the z-axis (as measured from the urethral orifice) within the imaging device 40, as shown in FIG. 7. The slit 131 in the imaging device 40 can allow the tubular guide channel 50 to be advanced to any distance within the imaging device 40. Once the appropriate locus in the z axis has been reached, the guide channel 50 can be rotated in the xy-plane for placement of aperture 80 of tubular guide channel 50 such that it matches the locus of slit 131 in the xy-plane.

Referring still to FIG. 7, a malleable needle 90 can be inserted through the tubular guide channel 50. The malleable needle 90 can have a tipped end 110 such that, as the malleable needle 90 is advanced out of the aperture 80, the tipped end 110 is able to engage and enter the tissue of interest, such as the prostate. The passage of the malleable needle 90 through aperture 80 can be facilitated by the negatively banked distal wall 60 of the tubular guide channel 50, as the slope can serve to gently advance the tipped end 110 out of the aperture 80. Moreover, the superelastic properties of the malleable needle 90 can allow the device to enter the tissue of interest with a predetermined curved arc toward the legion.

After the malleable needle 90 has been inserted, the stylet 110 can be withdrawn, leaving the malleable needle 90 in place, as shown in FIG. 8.

Referring still to FIG. 8, the length of core 101 can be introduced into malleable needle 90. The core 101 can be advanced such that the distal end of the core 101 is adjacent to the portion of the malleable needle 90 curved along the negatively banked edge 60 of the tubular guide channel 50. Although not shown, the distal end 130 of the core 101 can then be advanced to the tip of malleable needle 90 and through the edge of aperture 80 in tubular guide member 50, which, as discussed elsewhere herein can be set relative to the tissue of interest.

Referring again to FIG. 2, the core 101 can be rotated or spun relative to the longitudinal axis 181 of the core 101, for example, using a motor. Simultaneously, core 101 can be advanced out the distal end 121 of the malleable needle 90 and into the tissue of interest to a predetermined depth. The advancement may be actuated manually by a finger control on the drive device (not shown) or automatically using a motor programmed by precise digital input (not shown).

An image using MRI may be obtained at any point during the procedure to verify the proper position of the surgical instrument 111, including core 101. For example, an image may be obtained after the distal end 130 of the core 101 is advanced to the tissue of interest.

In one embodiment, when activated, the rotation of the core 101 can cause the sharp annular concentric member 114 to rotate through the tissue of interest and cut a sample. The core 101 can be removed from the malleable needle 90 in order to retrieve the sample from the distal end of the core 101. This removal can be accomplished, for example, by advancing an obturator coaxially through core 101 from the proximal to distal end and retrieving the sample from the distal end. Alternatively, a syringe can be attached to the luer on the proximal end 113 of the core 101, and the sample can be forced out with air or fluid. Alternatively, the drive unit can be connected to a vacuum apparatus such that the sample obtained by core 120 is sucked into a retrieval chamber. Other retrieval embodiments may be envisioned.

The size of the tissue sample obtained can be dependent on the diameter of core 101, typically measured in French size and for illustrative purposes between 3 French (1 mm) and 5 French (2 mm). Additionally, the size of the tissue sample can be determined by the depth of advancement of core 101 within the lesion. The size of the sample can also be determined by the tissue characteristics themselves.

The biopsy procedure as described herein can be repeated as necessary, such as one, two, or three times.

### Other Biopsy Or Therapeutic Procedures

Although the foregoing description discussed transurethral imaging and biopsy of the prostate, it will be appreciated that the surgical device can be used for other procedures as well.

For example, in one embodiment, the surgical instrument 111 can be used for transrectal biopsy of the prostate and biopsy of solid organs. The process can be the same as described hereinabove except that a sheath/needle assembly, such as a 13 gauge sheath needle can be advanced to the region of interest either by way of computer (using data obtained from MRI) or by hand, with verification of proper trajectory and end point established by repeat imaging. The needle can be removed and the sheath left in place. The imaging device 40 can be advanced through the sheath, which can then be removed.

In another embodiment, the surgical instrument 111 can be used for brain biopsy and for delivery of therapeutics. The process can be the same as described above except that a smaller gauge system may be utilized. A 16 gauge (1.6 mm) to 18 gauge (1.27 mm) sheath needle can be advanced into position using magnetic resonance stereotactic or three dimensional guidance techniques. The needle can be removed leaving the sheath in place and the imaging device 40 advanced through the sheath to the region of interest. Using surgical instrument 111 with tubular guide channel 20, MRI imaging of the central nervous system may be performed without traversing overlying critical neural tissue or vascular structures. In another embodiment, the surgical instrument 111 with an alternative to core 101 can be used. For example, using surgical instrument 111 and tubular guide channel 50, a curved semi-malleable or curved superelastic needle could be advanced to the point of interest of interest and a core biopsy using other cutting methodology. As an example, a needle with a curved scimitar shaped bevel could be employed to obtain a tissue sample by rotational and/or forward translational motion.

In another embodiment, surgical instrument 111 may be used as a platform to provide additional therapeutic techniques after a biopsy is performed by core 101 or a tissue cavity created by core 101. Through the lumen of core 101, brachytherapy seeds may be deposited in tissue. Alternatively, core 101 may be removed and brachytherapy seeds may be deposited directly through malleable needle 90. Alternatively, ablative devices such as radiofrequency ablation, cryoablation and other devices may be advanced through malleable needle 90 to provide local therapy.

In another embodiment, core 101 may be utilized for endoluminal procedures in arteries or veins using surgical instrument 111 for MRI endoluminal guidance. Alternatively, core 101 may be used with conventional radiographic guidance techniques. Core 101, for example, may be used to remove atherosclerotic deposits or blood clots from vessels using endoluminal imaging provided by MRI or using alternative imaging techniques known to those skilled in the art, including intravascular ultrasound (IVUS) and various radiographic angiographic techniques. The surface design of the annular concentric member 114 of core 101 may be varied as appropriate for the individual application. For example, a sharp saw tooth design may be used for cutting, resecting and ablative applications. Another example would be the use of an obtuse or undulating pattern for maceration of clot in arteries or veins.

In another embodiment, core 101 can be used as a recanalization device for nonvascular applications, such as obstructions to bile ducts, ureter, fallopian tubes or bowel. Such interventions may be performed with endoluminal MRI imaging guidance using surgical instrument 111 and tubular guide 50. Alternatively, core 101 may be employed for similar procedures using radiographic techniques with or without tubular guide 50.

In another embodiment, core 101 can be used in a form modified to function coaxially with an endoscope in order to sample tissue or perform other surgical techniques. Guide channel 50 similarly can be utilized in this embodiment to direct core 101 to a locus otherwise not accessible to the tip of the endoscope.

In another embodiment, core 101, with or without guide channel 50 can be utilized in a similar manner with a bronchoscope.

In another embodiment, core 101, with or without guide channel 50 can be utilized in a similar manner with a laparoscope.

In another embodiment, using the favorable characteristics of alloys such as nitinol for transmitting RF energy, core 101 may be utilized as a radiofrequency ablation device, since the distal edge represents the alloy without the overlying polymer wrap.

The foregoing descriptions are intended to illustrate various aspects of the present technology. It is not intended that the examples presented herein limit the scope of the present technology. It will be apparent to one of ordinary skill in the art that many changes and modifications can be made thereto without departing from the spirit or scope of the appended claims.

What is claimed:

1. A surgical instrument comprising:
a tubular member having a proximal end and a distal end;
at least one helical member concentric with the tubular member; and
a cutting portion protruding from the distal end of the tubular member.

2. The surgical instrument of claim 1, wherein there are at least two helical members, and wherein the at least two helical members are configured to reciprocate one another.

3. The surgical instrument of claim 1, wherein there are at least four helical members.

4. The surgical instrument of claim 1, wherein the at least one helical member comprises a nonferromagnetic material.

5. The surgical instrument of claim 4, wherein the nonferromagnetic material is an alloy of nickel and titanium.

6. The surgical instrument of claim 5, wherein the alloy of nickel and titanium is nitinol.

7. The surgical instrument of claim 1, wherein the tubular member comprises a polymer.

8. The surgical instrument of claim 7, wherein the polymer comprises polytetrafluoroethylene.

9. The surgical instrument of claim 1, wherein the cutting portion comprises the same material as the plurality of helical members.

10. The surgical instrument of claim 1, wherein the at least one helical member is embedded in the tubular member.

11. The surgical instrument of claim 1, further comprising a luer configured to attach the tubular member to a rotational drive device.

12. The surgical instrument of claim 1, further comprising a tubular imaging device coaxial with the tubular member.

13. The surgical instrument of claim 12, wherein the tubular imaging device is an MRI coil for receiving radiofrequency data.

14. The surgical instrument of claim 1, further comprising a tubular guide channel coaxial with the tubular member, wherein the tubular guide channel has a proximal end and a distal end.

15. The surgical instrument of claim 14, wherein the tubular guide channel comprises an aperture near the distal end.

16. The surgical instrument of claim 14, wherein the distal end of the tubular guide channel comprises a wall slanted at an angle.

17. The surgical instrument of claim 1, further comprising a malleable needle coaxial with the tubular member.

18. The surgical instrument of claim 17, wherein the malleable needle comprises a superelastic alloy.

19. A method of performing a biopsy comprising:
inserting a surgical instrument into a region of the body, wherein the surgical instrument comprises:
a tubular member having a proximal end and a distal end;
at least one helical member concentric with the tubular member; and
a cutting portion protruding from the distal end of the tubular member;
rotating the surgical instrument along an axis perpendicular to a radius of the tubular member such that the cutting portion cuts a sample of a tissue; and
withdrawing the surgical instrument such that the sample is removed from the region of the body.

20. The method of claim 19, further comprising inserting a malleable needle, wherein inserting the surgical instrument into the region of the body includes inserting the surgical instrument coaxially through the malleable needle.

21. The method of claim 20, wherein the malleable needle comprises a superelastic alloy.

22. The method of claim 19, further comprising inserting a tubular guide channel into the region of the body, wherein inserting the surgical instrument into the region of the body includes inserting the surgical instrument coaxially through the tubular guide channel.

23. The method of claim 22, wherein one end of the tubular guide channel comprises a wall slanted at an angle.

24. The method of claim 19, further comprising inserting a tubular imaging device into the region of the body, wherein inserting the surgical instrument into the region of the body includes inserting the surgical instrument coaxially through the tubular imaging device.

25. The method of claim 24, wherein the tubular imaging device is an MRI coil for receiving radiofrequency data.

26. The method of claim 19, further comprising imaging the region of the body to determine a location of the tissue.
